(19) United States
(12) Patent Application Publication
(10) Pub. No.: US 2026/0183457 A1
(43) Pub. Date: Jul. 2, 2026

(54) In-field Emergency Blood Transfusion System with Energy Efficient Pump

(71) Applicant: RBT Resources Inc., Yardley, PA (US)

(72) Inventors: Thomas M. LaMorte, Yardley, PA (US); John J. Sinisi, Warminster, PA (US); Jesse Sinisi, Warminster, PA (US)

(21) Appl. No.: 19/548,896

(22) Filed: Feb. 24, 2026

Related U.S. Application Data
(63) Continuation-in-part of application No. 19/066,131, filed on Feb. 27, 2025.
(60) Provisional application No. 63/560,154, filed on Mar. 1, 2024.

(51) Int. Cl. A61M 1/02; A61M 5/142; A61M 5/168; A61M 5/172; A61M 5/36

(52) U.S. Cl. CPC A61M 1/02; A61M 5/14232; A61M 5/16813; A61M 5/16831; A61M 5/172; A61M 5/36; A61M 2205/18

(57) ABSTRACT

A system and method of transfusing blood from a donor source to a patient. To facilitate the transfusion, a transfer tube is provided. One end of the transfer tube is connected to the donor source and the opposite end is intravenously connected to the patient. A pump assembly is provided that contains a pump and a microprocessor. The transfer tube is engaged with the pump assembly wherein the pump, when activated, acts upon the transfer tube to move blood. The microprocessor monitors the blood volume moved and automatically stops the pump once a predetermined volume of blood has been transferred. If the donor source is a person, the predetermined volume is between 400 milliliters and 450 milliliters. The flow rate of the pump is preferably 100 milliliters per minute.

### RELATED APPLICATION

This application is a continuation-in-part of U.S. patent application Ser. No. 19/066,131, filed Feb. 27, 2025, which claims the benefit of U.S. Provisional Application No. 63/560,154, filed Mar. 1, 2024. The entire disclosures of the above applications are incorporated herein by reference.

### BACKGROUND OF THE INVENTION

### 1. Field of the invention

In general, the present invention relates to blood transfusion systems that are used in emergency situations, such as during natural disasters or on a battlefield. More particularly, the present invention relates to transfusion systems with pumps and tubes that can transfer blood from a donor source to a person in need in a highly expedited manner.

### 2. Prior Art Description

The average adult male human body holds about 2000 milliliters of blood. If a person loses one-fifth of their blood, they are likely to go into hemorrhagic shock and lose consciousness. If a person loses one fourth of their blood, they are likely to die from exsanguination. Likewise, regardless of blood loss, if a person's blood pressure drops below 70/30 they are in danger of dying. In addition, even if the loss of blood does not cause death, the longer the state of hemorrhagic shock lasts, the more damage is caused to the body. Prolonged hemorrhagic shock can cause heart damage, brain damage, tissue loss and gangrene.

Blood loss to the point of exsanguination is typically caused from physical trauma, such as a gunshot wound, a shrapnel wound, or an impalement. The loss of blood is directly related to the location and size of the wound. In many instances, the wound can be bound to a degree where blood loss is manageable. However, if the patient has already lost too much blood, the blood must be replaced before the body passes into shock or passes from shock to death.

In cases of rapid blood loss, patients are typically treated using a blood transfusion. An intravenous line is inserted into the patient and a stored bag of blood or plasma is attached to the line. The blood or plasma then flows into the patient using the force of gravity. The flow of blood into the body using this system is typically around 70 ml per minute. This rate can be increased by using an intravenous line pump. However, even with such a pump, the maximum flow rate is around 80 ml per hour. Most intravenous line pumps are designed to introduce intravenous fluids, such as medicated saline solutions into a patient. Intravenous line pumps are designed to supply controlled volumes that are often measured in drops per minute, which translates to milliliters per hour. Intravenous line pumps are therefore designed for small flow rates and are not intended to maximize flow from a fluid source to the patient. Such prior art intravenous line pumps are exemplified by U.S. Pat. No. 9,677,555 to Kamen et al., U.S. Pat. No. 10,342,921 to Barnes et al., U.S. Pat. No. 5,399,166 to Laing and U.S. Patent Application Publication No. 2004/0064097 to Peterson.

Although such prior art intravenous line pumps can be considered transportable, they are still intended for use in a hospital or other stable environment, such as a field hospital, where there are clean conditions and access to external power. Such prior art intravenous line pumps are also designed for use on stabilized patients where the flow rate through the intravenous line can be kept under 300 ml per hour. Such prior art pump systems tend to contain drip chambers that only work when vertically oriented and therefore are poorly suited for battlefield use. In addition, such prior art systems cannot operate in battlefield conditions where there is no available external power, and the unit may be soaked with rain, snow, seawater, blood, and battlefield debris.

In many circumstances, a patient may need blood immediately and no stored blood or plasma is available. This is often the case on the battlefield. The transfusion is often conducted on an active battlefield while cramped behind limited cover. In such a circumstance, a medic may have to resort to a direct donor-to-patient blood transfusion, which is often referred to as an in-field blood transfusion. In an in-field blood transfusion, blood is directly transferred from a donor to a patient. The donor must remain available and stationary for the period of transfusion, which can be as long as a half hour per donor. This is highly impractical and often impossible for soldiers on a battlefield.

Devices that are designed for use on the battlefield or during a natural disaster must assume that power from a power grid is unavailable. Accordingly, such devices must be battery operated. However, although battlefield conditions and natural disasters do occur, they are rare. As a result, devices designed for use on the battlefield or during a natural disaster may sit unused for long periods of time. If the device is battery powered, the batteries must be charged or changed periodically. When a device is needed, it is not uncommon for the device to have old or otherwise partially drained batteries. This directly affects the usefulness of the device.

A need therefore exists for an improved system and methodology of performing an in-field blood transfusion, wherein the time required to transfer blood from a donor to a patient can be greatly reduced. A need also exists for an improved system and methodology of performing an in-field blood transfusion that can operate in any orientation and is robust enough to function without external power in a very wet and/or contaminated environment. Lastly, a need exists for an improved in-field blood transfusion device that is highly power efficient so it can operate for extended periods of time with used or undercharged batteries. This need is met by the present invention as described below.

### SUMMARY OF THE INVENTION

The present invention is a system and method of rapidly transfusing blood directly from a donor source to a patient. The donor source can be a living person or a prefilled bag of blood or blood plasma. To facilitate the transfusion, a transfer tube is provided. One end of the transfer tube is connected to the donor source. The opposite end of the transfer tube is intravenously connected to the patient.

A housing receives a section of the transfer tube and positions that section against a curved surface of a first arc length. A peristaltic pump is provided that moves a plurality of rollers. Within the peristaltic pump, each of the plurality of rollers is separated by a second arc length that is longer than the first arc length of curved surface. Each of the rollers periodically compress the section of transfer tube on the housing against the curved surface, therein moving said blood through said transfer tube.

The housing is a portable handheld unit that has a microprocessor controlled by a peristaltic pump. The portable housing holds at least one battery for powering the peristaltic pump, the microprocessor and other electronic components.

The microprocessor monitors blood volume moved by the pump and automatically stops the pump once a predetermined volume of blood has been transferred. If the donor source is a person, the predetermined volume is between 400 milliliters and 450 milliliters. The flow rate of the pump is preferably 100 milliliters per minute for a person-to-person transfusion and up to 300 milliliters per minute for a bag-to-person transfusion. As such, a transfusion from a human donor to a patient should take no longer than 4.5 minutes. A transfusion from a bag can take less than 2 minutes. The rapid rate of transfusion represents a significant advancement in the art that can save many patients from passing into hemorrhagic shock or passing from hemorrhagic shock to death.

### BRIEF DESCRIPTION OF THE DRAWINGS

For a better understanding of the present invention, reference is made to the following description of exemplary embodiments thereof, considered in conjunction with the accompanying drawings, in which:

FIG. 1 shows an overview of an exemplary embodiment of the present invention blood transfusion system;

FIG. 2 shows an overview of an exemplary embodiment of the present invention blood transfusion system shown in conjunction with a donor and a patient;

FIG. 3 shows the housing of the blood transfusion system in an open condition;

FIG. 4 shows the housing of the blood transfusion system in a closed condition;

FIG. 5 shows the engagement mechanics between the peristaltic pump and the transfer tube prior to roller engagement;

FIG. 6 shows the engagement mechanics between the peristaltic pump and the transfer tube during roller engagement;

FIG. 7 is a graph illustrating motor load verses time for the peristaltic pump corresponding to the conditions of FIG. 5 and FIG. 6;

FIG. 8 is a schematic of the major components contained within the pump assembly of the present invention;

FIG. 9 is a block diagram schematic showing the primary operational methodology utilized by the present invention;

FIG. 10 is a block diagram showing a preferred methodology of use for the present invention system; and

FIG. 11 shows an overview of an alternate exemplary embodiment of the transfer tube for use within the present invention blood transfusion system.

### DETAILED DESCRIPTION OF THE DRAWINGS

Although the present invention system and methodology can be embodied in many ways, only a few exemplary embodiments are illustrated. The exemplary embodiments are being shown for the purposes of explanation and description. The exemplary embodiments are selected in order to set forth some of the best modes contemplated for the invention. The illustrated embodiments, however, are merely exemplary and should not be considered limitations when interpreting the scope of the appended claims.

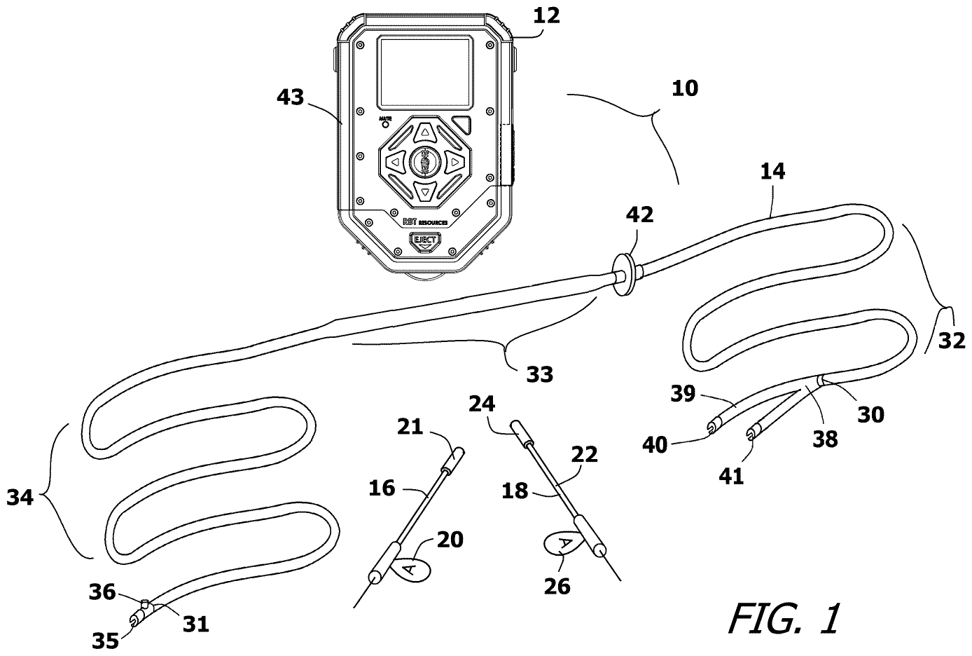

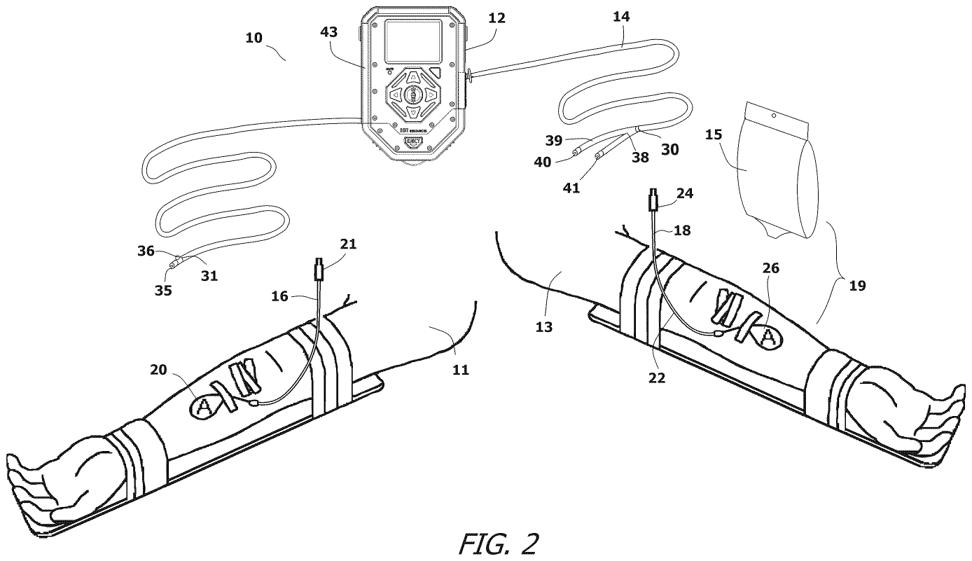

Referring to both FIG. 1 and FIG. 2, a first embodiment of an emergency blood transfusion system 10 is shown. The emergency blood transfusion system 10 contains a portable pump assembly 12, a transfer tube 14, and two intravenous ports 16, 18. These elements are transferred together in a unit package that is carried by a field medic or other medically trained personnel. The purpose of the emergency blood transfusion system 10 is to transfer a monitored volume of blood directly from a donor source 19 to a patient 11 in the shortest amount of time possible. The donor source 19 can be a person 13 or a prefilled bag 15 of blood or plasma.

To utilize the emergency blood transfusion system 10, the first intravenous port 16 is inserted into a patient 11 who is in need of blood. The first intravenous port 16 is firmly taped in place to prevent any inadvertent disengagement caused by the pressures generated by the emergency blood transfer system 10. The first intravenous port 16 is a standard IV port that has a first tube connector 21. A patient blood-type identifying graphic 20 is provided on the first intravenous port 16 for a purpose later explained. The blood-type identifying graphic 20 contains the letter(s) and polarity of the blood type along with a color scheme associated with that blood type. For example, many organizations including the American Red Cross® use of pink color code A+blood and light blue to color code O-blood.

Blood or plasma is drawn from the donor source 19. The donor source 19 can be a person 13 or a prefilled bag 15 of blood or plasma, as indicated in FIG. 2. If no preexisting bag of blood or plasma is available, the second intravenous port 18 is connected to a person 13. If multiple donors are available, each can be fitted with such an intravenous port 18. Each second intravenous port 18 preferably consists of a short tube segment 22 that terminates with a second tube connector 24. A donor blood-type identifying graphic 26 is provided for a purpose later explained. The identifying graphic 26 contains the letter(s) and polarity of the donor's blood type along with a color scheme associated with that blood type.

A transfer tube 14 is provided. The transfer tube 14 is a medical grade silicone tube or PTFE tube having a first end 30 and an opposite second end 31. The transfer tube 14 has a first section 32 that extends from the first end 30. The first section 32 has a preferred inner diameter of between 1.6 mm and 5.0 mm. The first section 32 of the transfer tube 14 extends into a second section 33. The second section 33 of transfer tube 14 is engaged by the portable pump assembly 12. The second section 33 has a length of between 15 cm and 22 cm. This provides enough length for engagement by the portable pump assembly 12. The second section 33 has an inner diameter that is larger than that of the first section 32. The purpose of the wider second section 33 is later explained. A third section 34 of transfer tube 14 extends from the second section 33 to the second end 31. The third section 34 of the transfer tube 14 has the same inner diameter as does the first section 32. The overall transfer tube 14 can have any length between one and three meters as measured between the first end 30 and the second end 31. The second end 31 of the transfer tube 14 terminates with a port connector 35. The port connector 35 is configured to selectively interconnect to the tube connector 21 on the patient's first intravenous port 16. The port connector 35 can have an optional venting valve 36 that enables the transfer tube 14 to be selectively vented. This enables air to exit the transfer tube 14 at the venting valve 36 during a priming procedure.

The opposite first end 30 of the transfer tube 14 can contain an optional Y-junction 38. Each arm 39, 41 of the Y-junction 38 terminates with a second port connector 40 that can be attached to the second tube connector 24 on the intravenous port 18 of the donor source 19. The Y-junction 38 enables the transfer tube 14 to be connected both to a first donor and/or to a bag of blood, plasma or saline. Alternatively, the Y-junction 38 enables the transfer tube 14 to be connected to a first donor and then to a second source just prior to the disconnection of the first donor. This enables a constant flow without needing to reprime the transfer tube 14. It also allows a bag of blood or plasma to be substituted for a donor and vice versa.

A one way valve 42 is positioned along the transfer tube 14 at a position in the first section 32 near the transition to the wider second section 33. The one-way valve 42 allows blood flow through the transfer tube 14 from the first end 30 toward the second end 31. The one-way valve 42 prevents any blood flow in the transfer tube 14 toward the first end 30.

The flow of blood through the transfer tube 14 is not controlled by gravity and is unaffected by gravity or orientation. Rather, the portable pump assembly 12 is used to pump the blood without directly contacting the blood. The portable pump assembly 12 has a containment housing 43. The containment housing 43 is designed for battlefield use and provides a hermetic barrier around the electronics and other working components in accordance with the casing standard outlined in military standard specification MIL C-4150J. The containment housing 43 is also crushproof by military standards. That is, the containment housing 43 is capable of maintaining integrity under 100 psi of pressure. Thus, the containment housing 43 can be stepped on or even run over by a vehicle without damage.

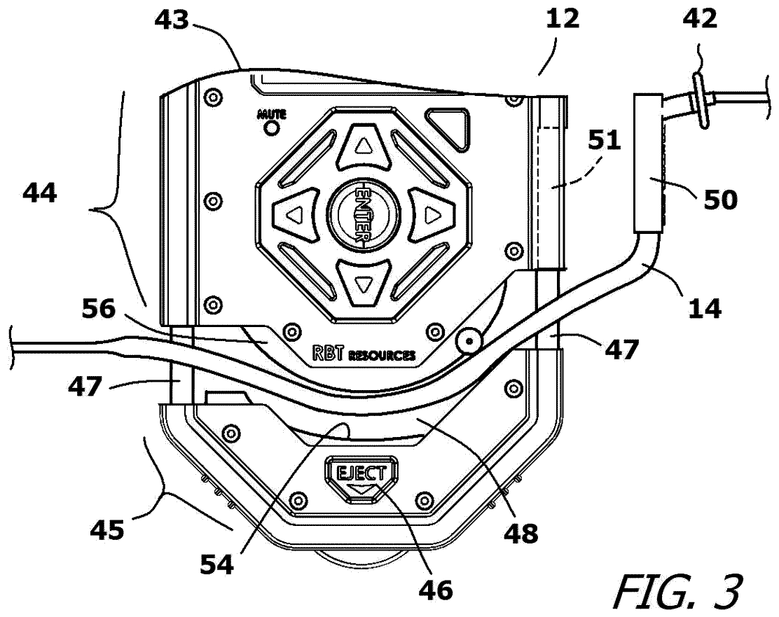

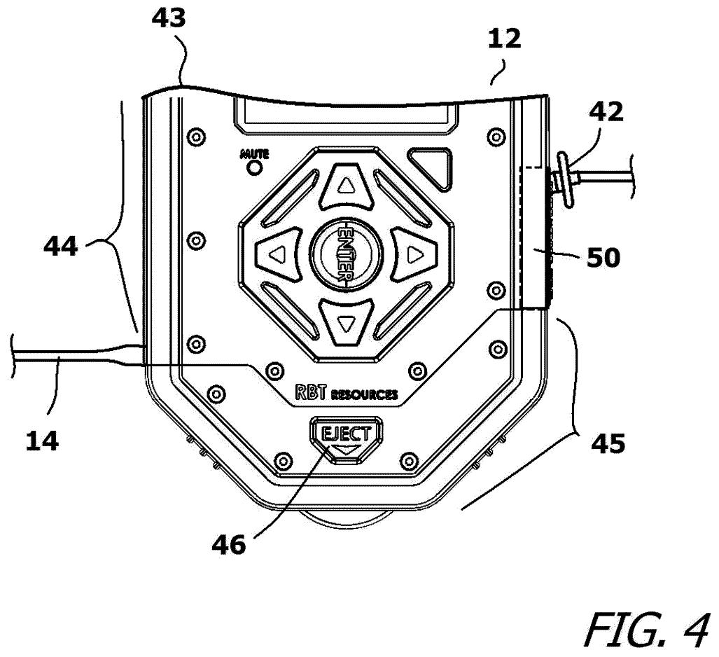

Referring to FIG. 3 and FIG. 4 in conjunction with FIG. 1, it will be understood that the containment housing 43 has a main section 44 and a lid section 45 that are separated to receive the transfer tube 14 and are joined to engage the transfer tube 14. The main section 44 of the containment housing 43 holds the electronics and electro-mechanical components of the portable pump assembly 12. The lid section 45 of the containment housing 43 has a release lock 46 that can be selectively engaged. The lid section 45 is joined to the main section 44 with slide rails 47. The slide rails 47 can be spring loaded. When the release lock 46 is engaged, the slide rails 47 extend and the lid section 45 moves away from the main section 44 of the containment housing 43. This creates a loading gap 48 between the lid section 45 and the main section 44. When the lid section 45 is manually pressed toward the main section 44, the slide rails 47 retract and the release lock 46 engages. This locks the lid section 45 in place atop the main section 44.

To engage the transfer tube 14 with the portable pump assembly 12, the lid section 45 of the containment housing 43 is opened to the configuration of FIG. 3. A removable locking cylinder 50 is provided in the structure of the containment housing 43. The containment housing 43 has a recess 51 that receives and retains the removable locking cylinder 50. The locking cylinder 50, when removed from the containment housing 43, is designed to engage the transfer tube 14. The locking cylinder 50 is attached to the first section 32 of the transfer tube 14 in an engagement area between the one way valve 42 and the start of the wider second section 33 of the transfer tube 14. The locking cylinder 50 engages the outside of the transfer tube 14 and does not kink or otherwise restrict flow through the transfer tube 14. Once the locking cylinder 50 is engaged with the transfer tube 14, the locking cylinder 50 is set into the recess 51 in the containment housing 43. This mechanically interconnects the transfer tube 14 with the containment housing 43 and prevents the transfer tube 14 from being pulled away from, or through, the portable pump assembly 12.

Once the locking cylinder 50 is connected to the transfer tube 14, the lid section 45 is moved to its open position, therein creating the loading gap 48. The locking cylinder 50 is connected to the recess 51 in the containment housing 43 and the transfer tube 14 is extended through the loading gap 48. The wider second section 33 of the transfer tube 14 extends through the loading gap 48. Once the transfer tube 14 is properly positioned, the lid section 45 of the containment housing 43 is closed around the transfer tube 14 and is locked with the main section 44.

When opened, the loading gap 48 has a width that is slightly larger than the widened diameter of the second section 33 of the transfer tube 14. As can be seen, the loading gap 48 is curved. The loading gap 48 is interposed between a curved static surface 54 at the bottom of the lid section 45 and the curved working surface of a specialized peristaltic pump 56 within the main section 44 of the containment housing 43. The curved static surface 54 at the bottom of the lid section 45 has a first arc length.

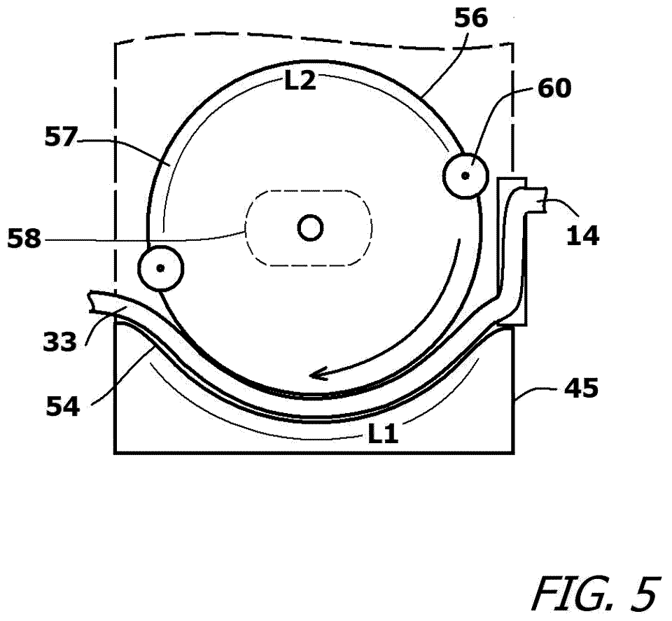

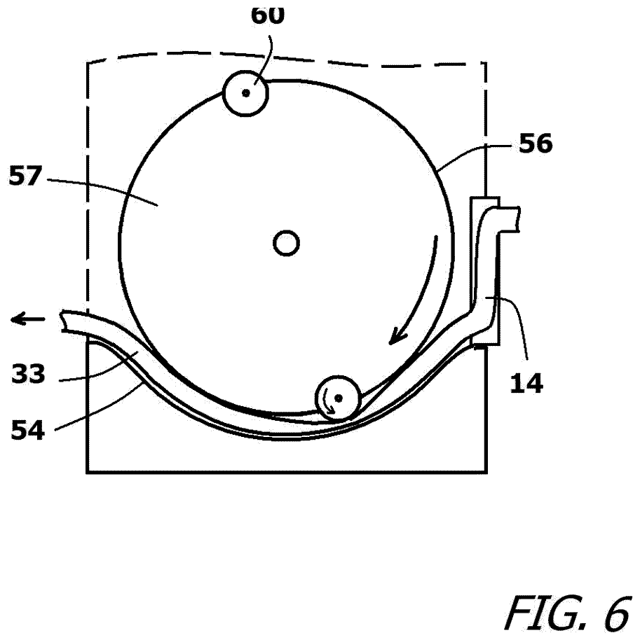

Referring to FIG. 5 and FIG. 6 in conjunction with FIG. 1 and FIG. 3, it can be seen that the specialized peristaltic pump 56 is within the main section 44 of the containment housing 43. The specialized peristaltic pump 56 has a primary drum 57 that is rotated by a high efficiency DC motor 58. The primary drum 57 has a diameter that fits within the containment housing 43. A plurality of contact rollers 60 are symmetrically mounted to the primary drum 57. The arc length L2 between the contact rollers 60 is greater than the arc length L1 of the curved static surface 54 at the bottom of the lid section 45. As a result, there is only one contact roller 60 on the specialized peristaltic pump 56 that passes along the curved static surface 54 at any one period in time. Furthermore, there are short periods of time when no contact roller 60 is engaged with the curved static surface 54.

As one of the contact rollers 60 passes under the curved static surface 54, the contact roller 60 compresses the wide second section 33 of the transfer tube 14 and displaces blood through the transfer tube 14. Blood contains a variety of cells, including red blood cells, white blood cells, and platelets, that can be damaged by mechanical forces and/or high pressures. By providing a widened second section 33 of transfer tubing 14 and engaging the transfer tubing 14 with only one contact roller 60 of the specialized peristaltic pump 56 at a time, the mechanical strains applied to the blood by the pumping action are greatly reduced. Furthermore, engaging the transfer tube 14 with only one contact roller 60 at a time prevents pressure from building up in the transfer tube 14 between the contact rollers 60 that can harm the blood. The result is little to no damage to the blood caused by the pumping action.

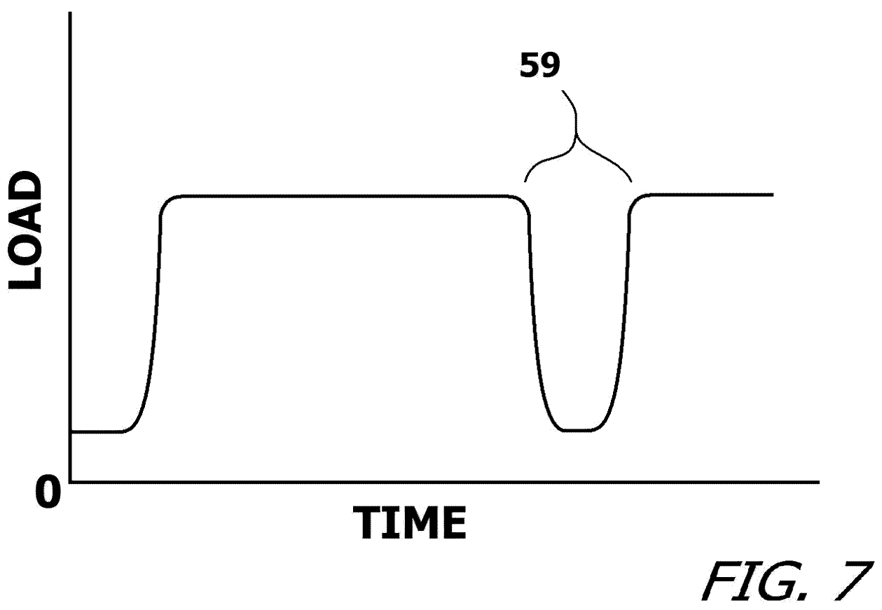

In addition to not harming the blood being pumped, the unique configuration of the specialized peristaltic pump 56 provides significant power consumption improvements to the portable pump assembly 12 that greatly reduces power draw and improves battery life. Referring to FIG. 7 in conjunction with FIG. 5 and FIG. 6, it can be seen that when no contact roller 60 is under the curved static surface 54, the transfer tube 14 is not compressed and the only work being done is the rotation of the primary drum 57. During these no pumping periods, the power requirements of the motor 58 are low and constant. As the contact roller 60 passes under the curved static surface 54, the second section 33 of the transfer tube 14 becomes compressed and the pumping action begins. The pumping action creates an increased load on the motor 58 and a corresponding increase in power requirements. Although the power draw increases the power requirements, the power requirements quickly level out and remain constant as the contact roller 60 moves under the curved static surface 54. There is only one increase in the power requirements since only one contact roller 60 passes under the curved static surface 54 at a time. This is different from other peristaltic pumps that contact tubes at multiple points when pumping and have multiple spikes in the power requirement profile. The constant draw of power created by using the widely spaced contact rollers 60 prevents the occurrence of high peak power spikes that cannot be met by undercharged batteries. Accordingly, by having only one contact roller 60 at a time engage the transfer tube 14, a constant, low power draw is created that enables the specialized peristaltic pump 56 to continue to operate even with undercharged batteries. Furthermore, there are brief periods 59 (FIG. 7) where no contact roller 60 contacts the transfer tube 14 and there is no significant load. This causes periodic rests in the power draw that give the batteries a chance to recover and therein greatly increase the charge life of the batteries. The result is a specialized peristaltic pump 56 that can operate for much longer periods of time with full batteries and will continue to operate with depleted batteries until the power available drops below that needed to turn the motor 58.

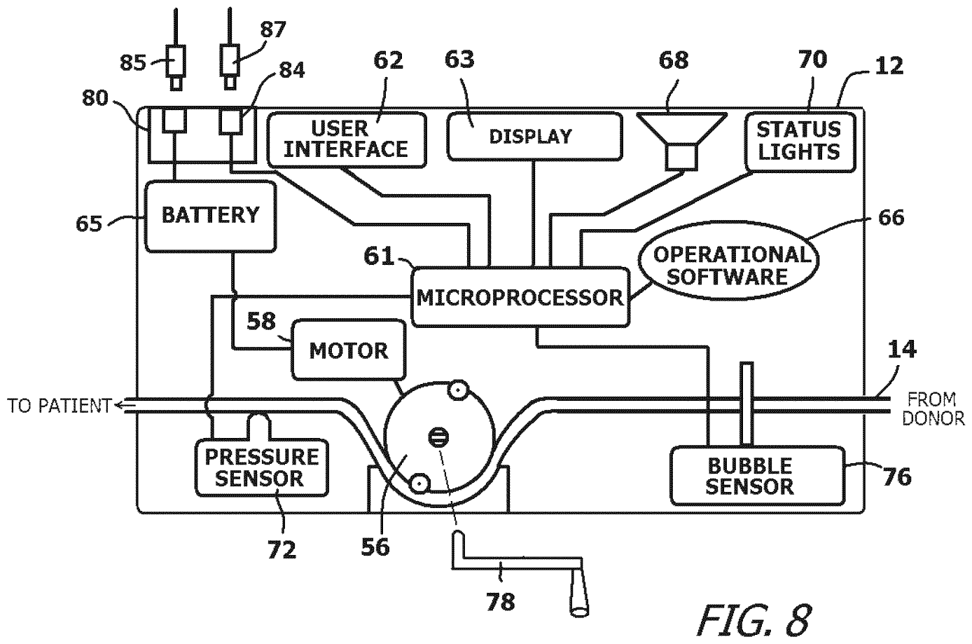

Referring to FIG. 8 in conjunction with FIG. 1 and FIG. 2, it can be seen that one or more batteries 65 are held within the portable pump assembly 12. The battery 65 is preferably a rechargeable battery of a type utilized by the U.S. Military. The rate that the specialized peristaltic pump 56 moves blood/plasma through the transfer tube 14 is controlled by a microprocessor 61 that is running operational software 66. An individual can interact with the operational software 66 using an interface 62 and display 63.

The motor 58 is connected to the microprocessor 61. The microprocessor 61 operates the motor 58 and uses the operational software 66 to monitor the rate at which the specialized peristaltic pump 56 is turning and the number of times that the specialized peristaltic pump 56 turns. As such, the microprocessor 61 can calculate the flow rate of blood/plasma through the transfer tube 14 and/or the volume of blood/plasma that has been transferred. These values can be viewed on a display 63 by engaging the user interface 62. The display 63 is capable of visually displaying data, instructions, and warnings. In addition to the display 63, a speaker 68 and status lights 70 are provided. The speaker 68 can audibly broadcast data, instructions, and alarms. The status lights 70 preferably contain a green light and a red light to quickly display the operational status of the system 10.

The pressure in the transfer tube 14 between the specialized peristaltic pump 56 and the patient can be monitored by the microprocessor 61. A pressure sensor 72 is optionally provided that presses against the transfer tube 14. The pressure sensor 72 is biased against the transfer tube 14. As the pressure in the transfer tube 14 changes, the resistance to the bias changes and the pressure within the transfer tube 14 can be determined without physically contacting the blood or plasma flowing through the transfer tube 14.

An optional bubble sensor 76 can also be provided. The bubble sensor 76 can detect bubbles and/or other occlusions or anomalies in the blood flowing through the transfer tube 14. If bubbles or occlusions are detected, an alarm can sound while the specialized peristaltic pump 56 can be stopped.

Optionally, the portable pump assembly 12 can have an input bay 80 that is protected by a waterproof closure 82. In the input bay 80 are input ports 84, 85 that lead to the microprocessor 61 and/or battery 65. The input port 85 for the battery 65 is a power lead that can engage a power cable to recharge the battery 65 or provide power in place of the battery 65. A least one data input 84 is provided that leads to the microprocessor 61. The data input 84 is used to connect the microprocessor 61 to an outside computer for program updates, data downloads, and diagnostic interrogations. The data input 84 can also be used to connect the microprocessor 61 to an auxiliary patient sensor 86, such as a pulse monitor or blood pressure monitor.

The operational software 66 can also monitor the operational rate of the specialized peristaltic pump 56 over time to calculate the volume of blood/plasma pumped during that time. The volume of blood/plasma pumped is stored by the operational software 66 for consideration by medical personnel attending to the patient. It is understood that during emergency use, the batteries 65 of the portable pump assembly 12 may drain from constant use. Replacement batteries or the ability to recharge the batteries may not exist. As such, the specialized peristaltic pump 56 preferably has the ability to receive a manual crank 78 so that the specialized peristaltic pump 56 can be manually turned when required. The crank can engage and turn either the motor 58 or the primary drum 57.

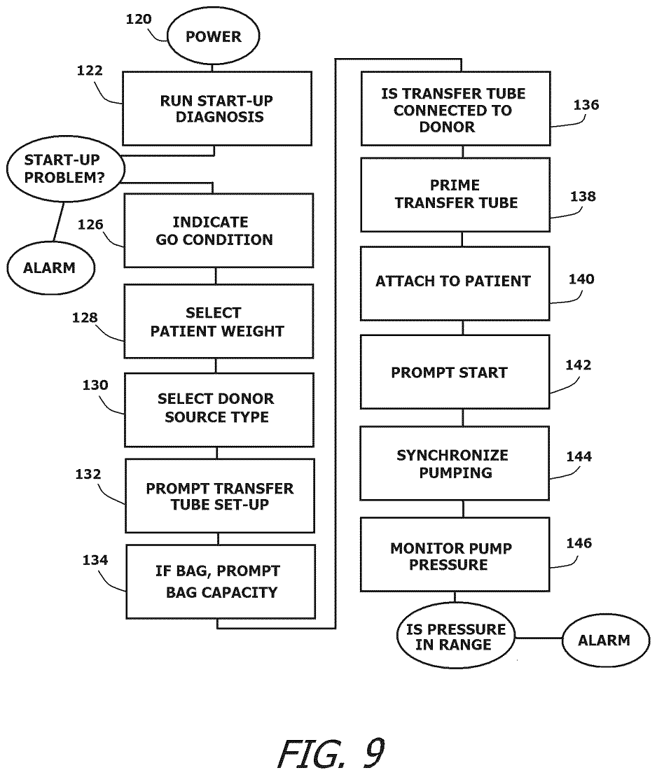

Referring to FIG. 9, in conjunction with FIG. 1 and FIG. 8, it can be seen that to utilize the present invention blood transfer system 10, a patient's bleeding rate is stabilized and a first intravenous port 16 is connected to the patient. See Block 90 and Block 92. A separate second intravenous port 18 is attached to a donor. See Block 94. The first intravenous port 16 and the second intravenous port 18 contain identifying graphics 20, 26. The identifying graphics 20, 26 can be color codes, number codes or other indicators of the blood type. In this manner, if there are many patients and many donors, a medic in an emergency situation can match the identifying graphics 20, 26 and quickly identify what donors are proper to donate to a specific patient. See Block 96.

Once a donor is matched to a patient 11, a transfer tube 14 can be connected to the donor. The transfer tube 14 is vented to allow the transfer tube 14 to fill with blood from the donor. Once primed with blood, the transfer tube 14 is connected to the first intravenous port 16 of the patient 11. See Block 98, Block 100, and Block 102. The transfer tube 14 is then engaged with the portable pump assembly 12. See Block 104. The portable pump assembly 12 is then activated. See Block 106. Once activated, the portable pump assembly 12 can draw blood from a donor at any rate that can be sustained by the donor. The preferred operating rate for an average adult male donor is 100 ml/min.

The microprocessor 61 in the portable pump assembly 12 monitors the flow of blood and automatically stops the flow once 450 milliliters of blood have been pumped. See Block 107 and Block 108. At the rate of 100 ml/min, the desired donation of 450 milliliters can be collected in only 4.5 minutes. If the flow of blood is pulsed in synchronization with the patient's heartbeat, the 450 milliliter of blood can be transferred in as little as 4 minutes. If blood/plasma is being drawn from a blood bag, the pumping rate can be raised to as high as 300 ml/min. This empties a 450 milliliter bag in under two minutes. This is multiple times faster than a traditional gravity-fed blood transfusion from a blood bag. This increase in blood transfer efficiency greatly reduces the chances that a person who has lost blood will enter hemorrhagic shock or die of exsanguination. Since the volume of blood is monitored and controlled, the donor will not be drained of a dangerous amount of blood. The microprocessor 61 will also monitor the flow rate and can sound alarms if the flow rate is too fast, indicating a disconnection or untreated hemorrhage, or too slow indicating a blockage. See Block 110 and Block 112.

As a donor approaches his/her maximum donation volume, the microprocessor 61 can display and/or sound a warning. This enables a medic to connect a different donor to the transfer tube 14 so that the initial donor can be safely disconnected. By connecting a second donor or a bag to the transfer tube 14 in this manner, no air gets introduced into the transfer tube 14 and no time is wasted repriming the transfer tube 14.

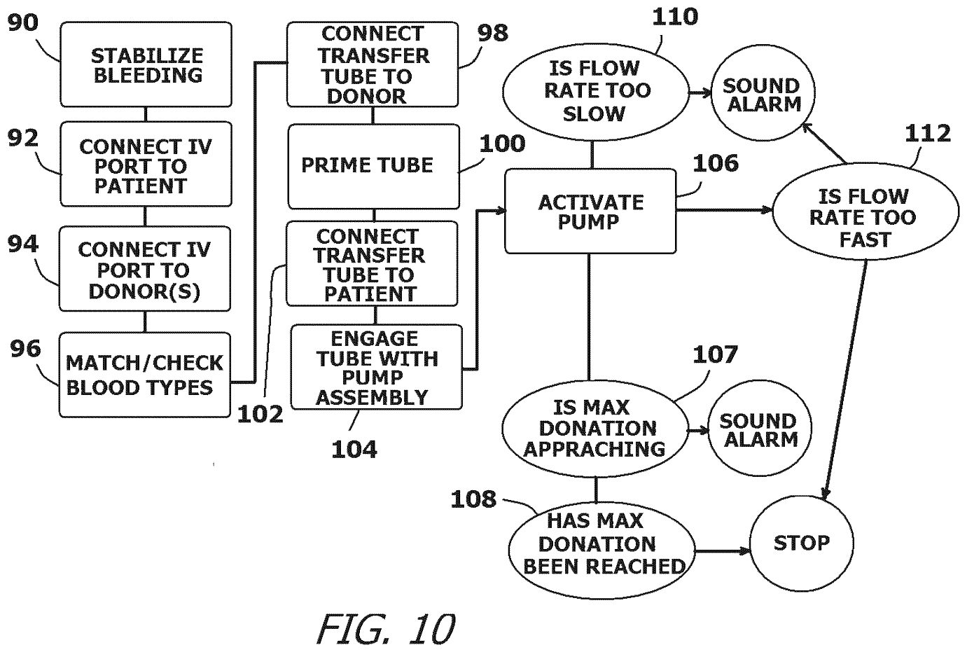

Referring to FIG. 10 in conjunction with FIG. 8 and FIG. 2, it can be seen that the microprocessor 61 runs the operational software 66. The inputs of an exemplary version of the operational software 66 are shown. As is indicated by Block 120, the emergency blood transfusion system 10 is powered on. Upon power up, the operational software 66 runs a rapid diagnosis that checks the status of the system and the power available in the batteries 65. See Block 122 and Block 124. If the system diagnosis is successful and there is power, a green light is displayed on the display 63. See Block 126. This may also be accompanied with some sort of an audible signal.

The medical professional using the emergency blood transfer system 10 is then prompted with a patient input prompt. In this prompt, the approximate weight of the patient is entered. See Block 128. This can be done using a menu selection presented on the display 63. For example, the prompt may indicate that the patient is 1. 100 lbs-140 lbs, 2. 141 lbs-180 lbs, 3. 181 lbs-220 lbs 4. Over 220 lbs. The size of a patient is proportional to the volume of blood in that patient and can be used to determine a maximum transfusion volume.

The next prompt is a prompt to indicate if the donor source will be a live donor or a prefilled bag. See Block 130. Once the donor source is selected, the user is prompted to connect the transfer tube 14 to a donor source and then insert the transfer tube 14 into the groove 55 on the housing 42. See Block 132. If the donor source is a person, the operational software 60 sets a limit of 450 milliliters of blood to be drawn. If the donor source is a bag, a second prompt is created that asks for the volume of the bag. See Block 134.

Once the transfer tube 14 is set, the operational software 66 generates a prompt asking if the transfer tube is connected to the donor source. See Block 136. If the prompt is answered in the affirmative, the operational software 66 prompts the user to open the venting valve 36 and the specialized peristaltic pump 56 runs for a few seconds to prime the transfer tube 14. See Block 138. Once the transfer tube 14 is primed, the venting valve 36 is closed. An over pressure is soon created in the transfer tube 14 that is detected by the pressure sensor 72 and the microprocessor 61 stops the specialized peristaltic pump 56.

The microprocessor 61 generates a prompt to attach the transfer tube 14 to the patient in need of blood. See Block 140. Once the transfer tube 14 is connected a “start” prompt is answered on the display 63 and the specialized peristaltic pump 56 starts pumping. See Block 142. If the microprocessor 61 receives biofeedback from the patient, the pumping can be synchronized to the heartbeat. See Block 144. This is a dynamically updated process since the heart may start beating faster or slower as blood supply in the patient increases. The pressure in the transfer tube 14 is monitored during pumping. See Block 146.

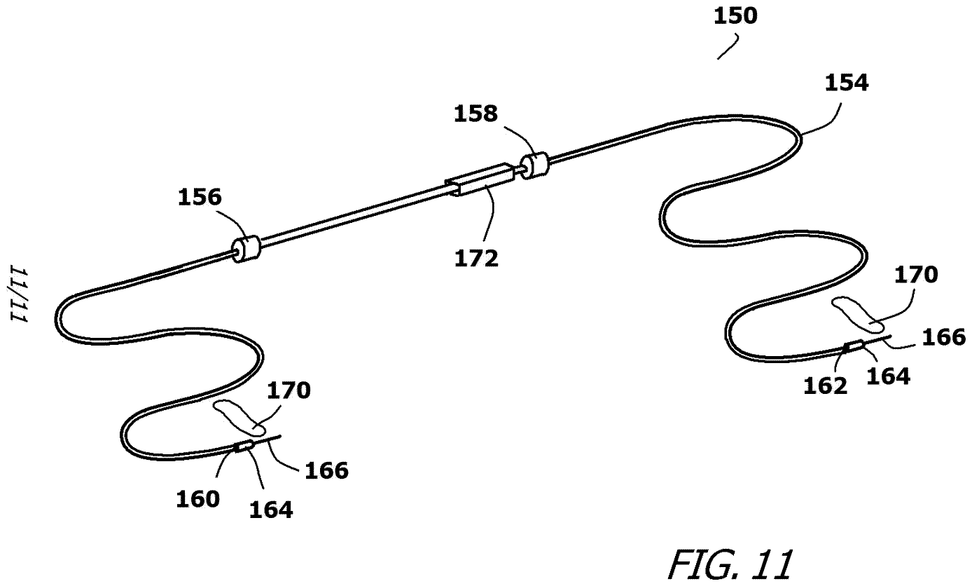

Referring now to FIG. 11, an alternate embodiment of the blood transfer tube 150 is shown. This blood transfer tube 150 is simplified and it can be carried in a sealed package by a medic in the field. The blood transfer tube 150 has an in-line filter 156 and a one way valve 158. The filter 156 and one way valve 158 prevent any clots or coagulations from passing into the patient and prevents any backflow into the donor.

The transfer tube 150 has a first end 160 and an opposite second end 162. Both ends 160, 162 terminate with connectors 164 that directly receive needle heads 166. In this manner, the patient and the donor need not be prepared with IV ports. Rather, the transfer tube 150 can be directly connected to a vein of the patient and an artery of the donor. Strips of tape 170 can be provided to hold the needle heads 166 in place and to identify blood type.

The transfer tube 150 also has a tube anchor 172 preset along its length. This enables the transfer tube 150 to be ready for use in an expedited manner.

It will be understood that the embodiments of the present invention that are illustrated and described are merely exemplary and that a person skilled in the art can make many variations to those embodiments. All such embodiments are intended to be included within the scope of the present invention as defined by the below claims.

What is claimed is:

1. A portable transfusion system for rapidly transfusing blood from a source into a patient, said system comprising:
a transfer tube that has a first end that connects to said source and a second end that connects to the patient;
a housing that receives a section of said transfer tube and positions said section of said transfer tube against a curved surface of a first arc length;
a peristaltic pump that moves a plurality of rollers, wherein each of said plurality of rollers is separated by a second arc length that is longer than said first arc length, and wherein said plurality of rollers periodically compress said section of said transfer tube against said curved surface, therein moving said blood through said transfer tube;
a microprocessor for controlling said peristaltic pump;
at least one battery for powering said peristaltic pump and said microprocessor.

2. The system according to claim 1, wherein a first section of said transfer tube leads into said section of said transfer tube received by said housing, wherein said first section has an inside diameter that is smaller than said section received by said housing.

3. The system according to claim 1, further including a tube lock that selectively engages said transfer tube.

4. The system according to claim 3, wherein said housing contains a recess for selectively receiving and engaging said tube lock.

5. The system according to claim 1, further including a one way valve on said transfer tube that prevents flow from said second end of said transfer tube toward said first end.

6. The system according to claim 1, wherein said peristaltic pump pumps said blood at a rate between 100 ml/min and 300 ml/min.

7. The system according to claim 1, wherein said microprocessor monitors blood volume transferred by said peristaltic pump and automatically stops said peristaltic pump once a predetermined volume of blood has been transferred.

8. The system according to claim 7, wherein said predetermined volume is between 400 milliliters and 450 milliliters.

9. The system according to claim 1, wherein said first end of said transfer tube terminates with a first intravenous connection.

10. The system according to claim 1, wherein said second end of said transfer tube terminates with a second intravenous connection.

11. The system according to claim 1, wherein said transfer tube includes a vent valve for venting air from said transfer tube.

12. The system according to claim 1, further including a pressure sensor connected to said microprocessor for monitoring pressure in said transfer tube, wherein said microprocessor sounds an alarm should said blood pressure fall outside a preselected range.

13. The system according to claim 1, further including a manual crank that is detachable from said housing, wherein said manual crank can engage and turn said peristaltic pump.

14. The system according to claim 1, wherein said housing has a main section and a lid section that can separate to create a loading gap, wherein said section of said transfer tube is positioned in said loading gap.

15. The system according to claim 1, wherein said curved surface is part of said lid section.

16. The system according to claim 1, wherein said main section and said lid section are connected by slide rails.

17. The system according to claim 1, wherein said second arc length is longer than said first arc length and produces periods where none of said plurality of rollers on said peristaltic pump compress said section of said transfer tube as said peristaltic pump operates.

18. A portable transfusion system for rapidly transfusing blood from a source into a patient, said system comprising:
a pump assembly that contains a pump, at least one battery for powering said pump, a microprocessor for controlling said pump, and a tube loading gap, wherein said pump, said microprocessor and said at least one battery are encased in a portable waterproof housing;
a transfer tube having a first end, a second end, wherein said transfer tube has a first diameter in a first section and a larger second diameter in a second section;
wherein said pump assembly receives said second section of said transfer tube in said loading gap and said pump engages said second section of said transfer tube in said loading gap.

19. The system according to claim 18, wherein said microprocessor monitors blood volume transferred by said pump and automatically stops said pump once a predetermined volume of blood has been transferred.

20. The system according to claim 18, wherein said pump is a peristaltic pump that moves a plurality of rollers, wherein each of said plurality of rollers is separated by an arc length, and wherein said plurality of rollers periodically compress said transfer tube, one of said plurality of rollers at a time, in said loading gap, therein moving said blood through said transfer tube.
